(19) United States
(12) Patent Application Publication
(10) Pub. No.: US 2008/0108909 A1
(43) Pub. Date: May 8, 2008

(54) PREDICTION OF CLINICAL OUTCOME USING LARGE ARRAY SURFACE MYOELECTRIC POTENTIALS

(75) Inventors: Steven I. Reger, Solon, OH (US); Vinoth K. Ranganathan, Parma, OH (US); Robert S. Butler, Shaker Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/865,238

(22) Filed: Oct. 1, 2007

Related U.S. Application Data
(60) Provisional application No. 60/848,706, filed on Oct. 2, 2006.

(51) Int. Cl. A61B 5/04

(52) U.S. Cl. 600/546

(57) ABSTRACT

Methods and systems are provided for non-invasively predicting the outcome of facet nerve block procedures on patients experiencing lower back pain. A sensor array is configured to capture a set of large array surface electromyographic (LASE) data from a patient. A feature extractor computes at least one value representing the patient from the captured LASE data. A classifier selects one of a plurality of outcome classes for the patient according to the computed at least one value.

### RELATED APPLICATION

This application claims priority from U.S. Provisional Application No. 60/848,706, filed Oct. 2, 2006, the subject matter of which is incorporated herein by reference.

### FIELD OF THE INVENTION

The present invention relates to a system and method for predicting outcomes of clinical procedures and, in particular, is directed to systems and methods for non-invasively predicting the outcome of facet nerve block procedures on patients experiencing lower back pain.

### BACKGROUND OF THE INVENTION

The presence of low back pain (LBP) is a widely occurring health care problem with an annual incidence of five percent and a lifetime prevalence of sixty to ninety percent in the general population. It is also one of the most expensive disabilities in our society costing more than fifty billion dollars annually. Lumbar Zygapophyseal (facet) joints have been implicated as one of the main causes of LBP with a reported prevalence of fifteen to fifty-two percent in patients with LBP. Nerve blocks (i.e., highly controlled anesthetic block of the medial branch of the nerve innervating the target facet joint) are used for diagnosis of lumbar facet joint pain and they can be potentially therapeutic. Patient reported pain relief of fifty percent or more following a precise nerve block confirms the facet joint as the source of pain.

Studies requiring the most stringent standards for relief of symptoms after a diagnostic block report a 4.0-7.7% prevalence rate of facet joint pain among chronic LBP patients. Studies using double blocks requiring 50% pain relief report prevalence rates of 9-15%. Numerous other studies using a single diagnostic block report prevalence rates from 16-75%. Due to this high degree of variability in the prevalence rate, studies have tried to determine predictors of facet joint pathology. Revel and colleagues compared ninety maneuvers and symptoms associated with facet joint pathology and reported 81.8% sensitivity and 77.8% specificity for four symptoms out of seven predictors they have found important. Another issue is the high rate of false positives—i.e. patients who do not have facet joint pathology, but experience pain relief due to the anesthetic effect of the nerve block procedure. Manchikanti et al, 2000, in clinical examination, found false positives in up to 25% of the patients with suspected facet syndrome.

There is also a lack of consensus about the assessment and treatment of low back problems with significant variations in the use of diagnostic tests and interventions for evaluating low back pain (Deyo et al, 1991). A major source of frustration for clinicians has been the fact that no reliable means exist to document a clinical diagnosis of lumbar facet joint pain without the use of invasive techniques. Fluoroscopically guided blocks of the joints constitute the only available standard to correlate with any clinical or radiographic test for facet joint pain. If the true prevalence of facet joint pain was 40-75%, as initially reported, a clinical profile may not be crucial because all LBP patients would warrant investigation for this disorder. However, if the true prevalence is only 5-15%, a clinical profile becomes important to prevent indiscriminate use of diagnostic and/or therapeutic blocks.

### SUMMARY OF THE INVENTION

In accordance with one aspect of the present invention, a computer program product is provided for non-invasively predicting the outcome of facet nerve block procedures on patients experiencing lower back pain. A sensor array is configured to capture a set of large array surface electromyographic (LASE) data from a patient. A feature extractor computes at least one value representing the patient from the captured LASE data. A classifier selects one of a plurality of outcome classes for the patient according to the computed at least one value.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a classification system for predicting the effectiveness of a clinical procedure on patients experiencing pain, in accordance with an aspect of the present invention;

FIG. 2 illustrates an exemplary placement of sensors in a fixed array;

FIG. 3 illustrates a schematic diagram showing a portion of the boxes formed by the electrode array;

FIG. 4 illustrates an exemplary methodology for training and verifying a classification system in accordance with an aspect of the present invention; and

FIG. 5 illustrates a computer system that can be employed to implement systems and methods described herein, such as based on computer executable instructions running on the computer system.

### DESCRIPTION OF EMBODIMENTS

In accordance with an aspect of the present invention, objective methods have been developed for determining the potential effect of diagnostic interventions and proposed treatment for pain control for a patient from large array surface electromyographic (LASE) data. By large array surface electromyographic data, it is meant to encompass any data that is produced from a reasonably large (e.g., >20) number of surface electrodes configured to measure electrical potential over a desired muscular structure. The surface electrodes can be spread over various portions of the body, but, the electrodes are maintained in groups of electrodes representing individual regions of the body, such that a given region is represented by a grouping of electrodes. In accordance with an aspect of the present invention, the groups of electrodes can be arranged symmetrically relative to a midline of a patient's body such that similar muscle groups on opposite sides of the body (e.g., left and right Latissimus Dorsi, left and right deltoids, etc.) are represented by corresponding groups of electrodes. This allows symptomatic regions of the body to be compared with a presumably normal control on the other side of the body, providing additional context for the analysis of the LASE data. LASE data can be correlated with clinical signs and symptoms to produce noninvasive, reproducible, myoelectric data and color-coded maps of the activity of muscles in an area of interest.

For example, in a recent clinical study, it has been demonstrated that the LASE technology can be used to accurately differentiate and classify 95.5% of patients with acute low back pain and 99.4% of subjects in the healthy groups. Much of the following discussion deals with an exemplary implementation of the invention for diagnosing lower back pain to determine the appropriateness of various interventions for facet joint syndrome. It will be appreciated, however, that the systems and methods described below can be applied generally to LASE data generated at any portion of a living body to determine appropriate diagnoses and interventions for any of a number of disorders. For example, the system can be applied to determine the sources of pain in the upper back, neck, and extremities of a patient.

The study has shown that patients with the facet joint syndrome will have different LASE patterns than those without this syndrome before the injection for medial nerve branch block and that this difference in LASE pattern can be used to predict the level of pain relief expected from the nerve block. The lumbar facet joint is a small synovial joint comprised of the inferior articulating surface of the vertebrae above and the superior articulating surface of the vertebrae below. There are two facet joints for each two articulating vertebrae. The ligamentous joint capsule allows for flexion, extension and lateral flexion. Rotation is limited in the lumbar spine due to the orientation of the joint surfaces. The facet joint is innervated by the medial division of the posterior primary ramus of the spinal nerve. Because the lumbar spine is polysegmentally innervated, a selective nerve root block will not identify the precise source of pain when the facet joint is suspected.

In a facet nerve block diagnostic procedure, a small amount of local anesthetic and steroid is injected in the region of the medial branch of the posterior primary ramus of the spinal nerve supplying the facet joint thought to be the source of pain. The effectiveness of the nerve block is determined by the pain relief that occurs after the short acting local anesthetic wears off (typically 2-3 hours) but while the effects of the long-acting steroid are still present (several days). The amount of pain relief varies between patients. If a patient reported at least a 50% pain relief (e.g., measured by a score between 1-10 on the VAS, where 1=no pain and 10=worst pain) following the initial nerve block, the facet joint was likely the source of pain. This level of pain relief is the threshold used clinically to make treatment decisions and it is determined based on the patient's recollection of the maximum relief.

Although the clinical effect of the nerve block is temporary, a small percentage of patients, referred to herein as the permanent improvement group, will report continued relief from the first injection, and that may preclude further treatment. Patients with temporary relief after the first injection, referred to herein as the temporary improvement group, can be scheduled for further treatment, such as a second nerve block in two to three weeks and a radio frequency ablation (RFA) of the nerve that provides semi-permanent relief. Patients who have a source of pain other than the facet syndrome, referred to as the no improvement group, will get little or no relief from the block and can be referred for alternative diagnosis and treatment. The differences in the pre-intervention LASE data obtained from these different nerve block outcome groups can be used to reliably predict the clinical outcome following the facet nerve block.

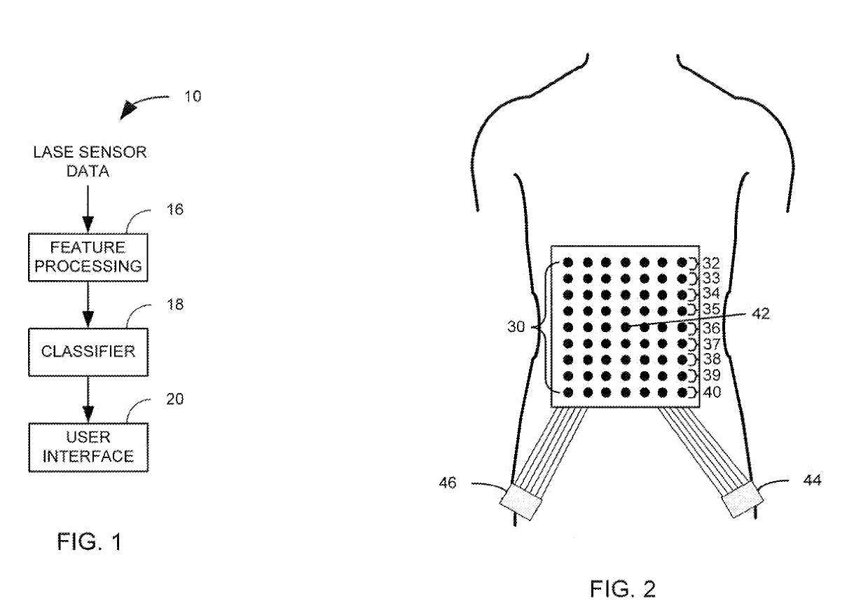

FIG. 1 illustrates a classification system 10 for predicting the effectiveness of a clinical procedure on patients experiencing pain in accordance with an aspect of the present invention. In accordance with an aspect of the present invention, large array surface myoelectric (LASE) sensor data representing the patient can be provided to a feature processing component 16. The LASE sensor data can be obtained from any appropriate portion of a patient's body, including the neck, back, and extremities of the patient, for evaluating the effectiveness of any of a number of possible treatments. It will be appreciated that as used herein, a “large array surface myoelectric sensor” refers to any of a number of encompass any configuration of a reasonably large (e.g., >20) number of surface electrodes configured to measure electrical potential over a desired muscular structure.

The feature processing component 16 computes at least one value representing the patient from the captured sensor data. The computed feature values represent at least one feature that is useful for distinguishing among the plurality of output classes associated with the classification system. It will be appreciated that any of a number of features can be derived from the provided feature data for this purpose. For example, a plurality of appropriate features can be selected empirically.

In an exemplary implementation, each scan is screened for artifacts, high noise levels, bad scans and bad electrode array. In the exemplary implementation, a 7×9 electrode array can be evaluated as a grid of 6×8 “boxes”. It will be appreciated, however, that the systems and methodologies described herein can be scaled to an electrode array of any size. Further, the array can be divided into multiple strips for positioning the array over multiple, spatially remote locations on the body. The “boxes” defined within the divided area can be selected as to maintain symmetry across an area of interest, such that all boxes that are not on a midline of the area of interest have a corresponding box on the other side of the midline. In an exemplary implementation, the midline is aligned with the patient's body. Accordingly, one box might represent a region of the patient's upper left arm. A corresponding second box would represent a portion of the patient's right arm. By maintaining symmetric regions across the midline of the patient's body, the behavior of corresponding muscle groups can be compared. Thus, a first portion of the patient's body, where no symptoms are present can be compared to a symptomatic portion of the patient's body to determine if differences in function are present.

To avoid double counting of voltage measurements, the measurements associated with an individual box consist of voltage readings for the left side of the box on the left half of the array or on the right side of the box when the boxes are on the right side of the array, the bottom of the box, and both of the diagonal readings. Several boxes can also be defined along the midline of the array and in the top most row of the array that contain only one electrode pair. In an exemplary implementation, four electrode pairs in each of forty-eight boxes are averaged, for example, as a root mean square (RMS), to produce a value for the box. The averaged forty-eight boxes and fourteen single electrode pair boxes from the top row and vertical midline of the array are adjusted to account for differences in patients BMI.

In an exemplary implementation, the system 10 can be used to predict clinical outcomes for a facet nerve block. It will be appreciated, however, that the system can be used to identify and predict outcomes for other clinical treatments, including treatment of myogenic, discogenic, and osteogenic pain within the lower back, as well as pain in other locations within the body. Three test positions, representing increasing levels of minimal low back stress, were utilized including standing upright (Standing), standing with 20 degrees of trunk flexion (Flexion), and standing upright with arms extended forward holding a small weight of three pounds in each hand (Weighted). Three scans of the EMG electrode array were performed at each postural position with thirty seconds rest between scans and sixty seconds rest between postural positions. The scans were stored digitally for analysis and visual display.

In an exemplary implementation, a correlation analysis can be performed between BMI-adjusted, box-averaged data from each patient and the grand means from each diagnostic class for each patient position.

The equation for the correlation coefficient is

$${ρ}_{x , y} = \frac{Cov ( X , Y )}{{σ}_{x} \cdot {σ}_{y}}$$

where −1≦ρ_{x,y}≦1 and

$$Cov ( X , Y ) = \frac{1}{n} {\sum }_{j - 1}^{n} ( {x}_{j} - {μ}_{x} ) ( {y}_{j} - {μ}_{y} )$$

In the above equations, Cov(X,Y) is the covariance of individual box data from the patient (X) and the corresponding grand mean from the patient diagnostic classes (Y) across all of the boxes. μₓ and μ_{y} are expected values and σₓ and σ_{y} are standard deviations. The covariance of two random variables X and Y is defined as the expected value of the product of the deviations of X and Y from their respective means. The covariance expresses the way X and Y trend with one another. For example, if large positive deviations of X from its mean are associated with large positive deviations of Y from its mean and large negative deviations of X from its mean are associated with large negative deviations of Y from its mean, the covariance will be positive. The covariance provides a measure of association or relatedness between two variables.

It will be appreciated that the boxes formed from the array can be divided into regions, with correlation coefficients being calculated separately for each region and each diagnosis class having a set of values for each region. The correlation value calculated for each region can be compared into one or more common metrics for analysis as described below. In one implementation, the data from the array can be separated into the left side of the array and the right side of the array, and the data for each side can be compared to respective left side and right side data for each diagnosis class and patient position.

The correlation coefficients for each position and region can then be transformed into z parameters using Fisher z transform to allow for the computation of a common metric. This transform converts the correlation coefficients into values that can be treated as averages. This means the z values can be multiplied by their respective degrees of freedom and the results used to compute grand averages. The equation for the Fisher transformation is:

$${z}^{′} = \frac{1}{2} ln ( \frac{1 + x}{1 - x} )$$

where x is the correlation coefficient.

Each z has a variance expressed as ρ²=1/(n−3), where n is the number of degrees of freedom. The z parameter has the same number of degrees of freedom as the number of points in the correlation (28 per side). The grand average of a group of averages is computed by multiplying each average by its respective degrees of freedom, summing the individual products, and dividing this sum by the total number of degrees of freedom. The transformed correlation coefficients for each group can be averaged for various combinations of positions. It will be appreciated that a single position may not always accurately classify the patients into one of the three groups. Accordingly, different combinations of the three positions can be utilized. In the described example, a combined correlation coefficient can be formed by averaging the transformed correlation coefficients from all three positions for each region. The combined correlation coefficients from each region can be averaged and normalized across the three groups to provide a prediction probability. These prediction probabilities can be used as feature values to classify patients into their respective groups.

A classification system 18 classifies the feature data determined from each patient into one of a plurality of diagnosis classes. It will be appreciated that any of a number of classification techniques can be utilized in classifying the patient data, including neural networks, statistical classifiers, support vector machines, and self organizing maps. In an exemplary implementation, the classifier can comprise a rule based classification system that categorizes the patient according to a series of logical rules. For example, an outcome group with the highest prediction probability can be selected as the prediction regarding the clinical outcome. It will be appreciated, however, that a classification determination in accordance with an aspect of the present invention can include other features derived from the sensor data and a more complex classification algorithm.

The results of the classification can be provided to a user interface 20. The user interface 20 can include software allowing the system to interface with one or more external hardware or software components such that the user can view and edit the outputs of the feature extractor and the classification system 18. It will be appreciated that the user interface 20 can be utilized both in training and using the classifier 18.

FIG. 2 illustrates an exemplary placement of sensors in a large array surface myoelectric (LASE) sensor array 30. The exemplary placement shows the LASE sensor array positioned on the lower portion of a human back, but it will be appreciated that this is merely exemplary and that the systems and methods described herein can be applied to other positions on the human body or to non-human living bodies. In one implementation, the illustrated array can be part of a Computerized Electromyographic Reconstruction of Spinal Regions system (CERSR) developed by SpineMatrix of Cleveland, Ohio. In the exemplary implementation, the CERSR instrument can include a Windows based NT operating system with a Pentium II central processing unit plus a 64-channel analog to digital converter (2000 Hz) and 62 amplifiers (frequency bandwidth of 30 Hz to 150 Hz). The surface myoelectric potentials can be collected from a fixed array of 63 pre-jelled, self-adhesive, silver-silver chloride electrodes (1 cm in diameter, spaced 3.0 cm apart, center to center) mounted on a plastic sheet.

In the illustrated example, the LASE sensor array 30 of discrete electrodes cover a 19.0 cm by 24.5 cm skin area. The array includes sixty-three electrodes arranged in nine rows 32-40 of seven electrodes each. The center or reference electrode 42 in the array can be positioned 6 cm above the spinous process of L4 vertebra. The output of the plurality of electrodes can be provided to one or more output ports 44 and 46 that are electrically connected to the plurality of electrodes. The output ports 44 and 46 can be connected to a classification system and database (not shown) such that the sensor data can be stored and evaluated. For example, a given LASE image display can contain sixty-three dots, one for each electrode in the array, and two hundred six nearest neighbor electrode pair combinations, represented by colored bars connecting the adjacent electrodes on the image.

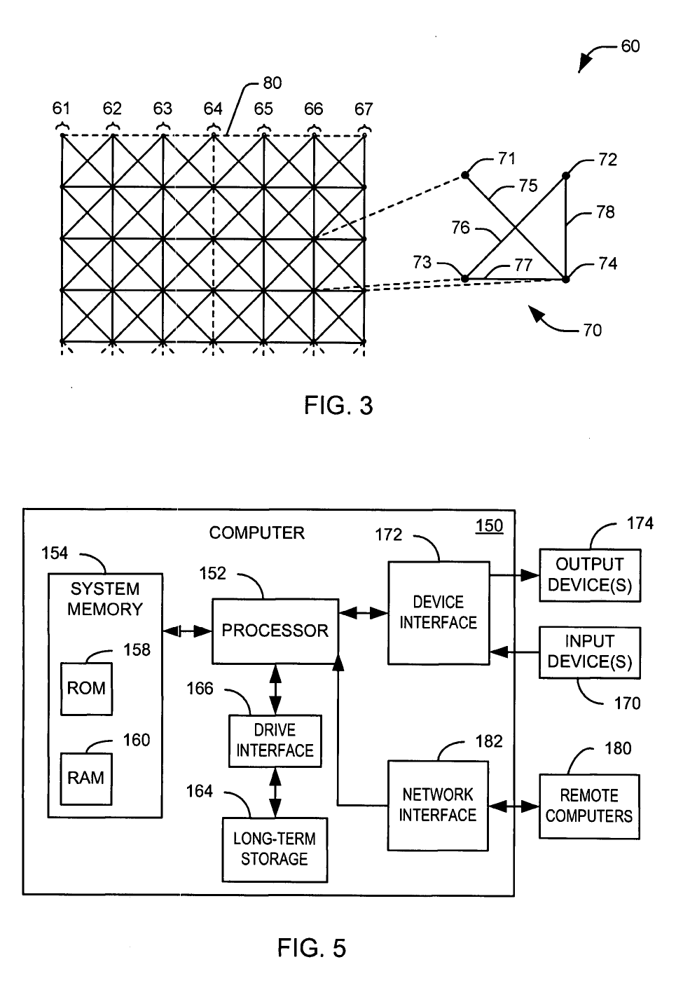

FIG. 3 illustrates a exemplary schematic diagram 60 showing a portion of the “boxes” selected within the electrode array in one implementation. The array includes seven columns 61-67 of electrodes including a left edge 61, a vertical midline 64 and a right edge 67. A given box (e.g., 70) is bounded by four electrodes 71-74 represents four electrode pairs 75-78. A first electrode pair 75 represents a potential difference between a top-left electrode 71 and a bottom right electrode 74. A second electrode pair 76 represents a potential difference between a top-right electrode 72 and a bottom left electrode 73. A third electrode pair 77 represents a potential difference between a bottom-left electrode 73 and a bottom right electrode 74. A fourth electrode pair 78 will be defined differently depending on which side of the midline 64 upon which it falls. On the left side of the midline, the fourth electrode pair 78 represents a potential difference between a top-left electrode 71 and a bottom left electrode 73. On the right side of the midline, the fourth electrode pair 78 represents a potential difference between a top-right electrode 72 and a bottom right electrode 74. This organization is utilized to improve symmetry and correlation analysis. Additional boxes, containing only one electrode pair, can be defined along the midline 64 of the array and the top most row 80. Basically, each pair of neighboring electrodes along these lines is considered as an electrode pair. Therefore, of two hundred six electrode pairs, one hundred ninety-two pairs are assigned to the forty-eight boxes, with four pairs per box, and the remaining fourteen electrode pairs are defined as individual boxes along the mid-line of the array and the top most row.

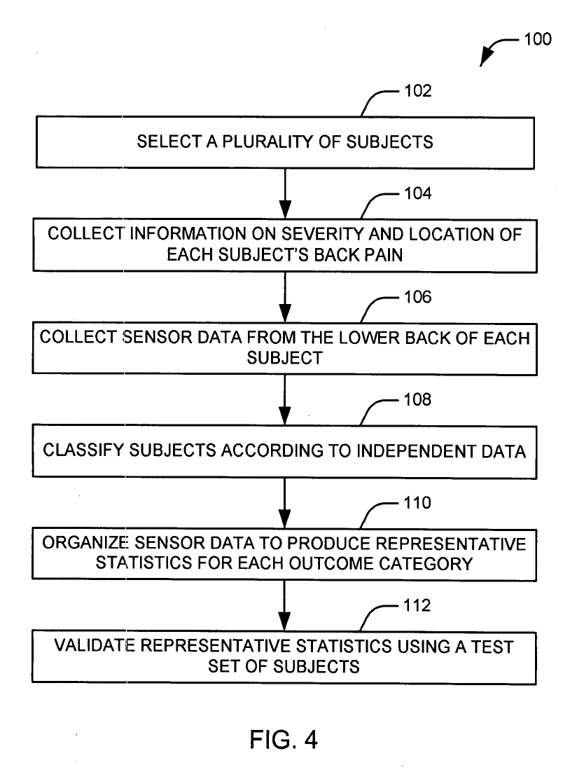

FIG. 4 illustrates an exemplary methodology 100 for training and verifying a classification system in accordance with an aspect of the present invention. At step 102, a plurality of subjects are selected. In one example, forty patients (Age: 56.8±14.8 yrs, 20 females, 32.325±7.98 BMI) with low back pain were selected as a training and testing set for the classification system. Large array surface electromyographic (LASE) data was collected from the patients by a Computerized Electromyographic Reconstruction of Spinal Regions system. The subjects that were selected all had a diagnosis of facet syndrome lower than T-10, were between eighteen and eighty years of age, and were scheduled for their first lumbar facet joint medial branch nerve block. Potential subjects were excluded if they had had prior spine surgery lower than T-7, diabetes, or a thyroid disorder. The patients were provided with clinical treatment independently of the study.

At step 104, information is collected from each subject to determine the severity and location of their pain. In one example, study patients completed the Oswestry Questionnaire and the Cleveland Clinic Foundation's Spine Questionnaire Part I, (PWO 5862, Rev. 2/99) including the VAS for Pain, pain drawings, and the Roland Back Pain Assessment. A print out of the electrode array location on the patient's back was provided and the patient was asked to circle the painful areas before and after the facet joint nerve block. It was assumed the pain level would remain constant throughout the 15 minutes of data collection. The Institutional Review Board at the Cleveland Clinic approved the protocol and all subjects provided their informed consent prior to entering the study.

It will be appreciated a desired sample size for training a given classifier will depend on the number of classes, the distribution of the population among the classes, and the features utilized to distinguish among the classes. In one example, the population sample size for training was obtained from the statistical power of a data analysis methodology associated with the classification system for detecting a significant difference between two proportions. The first proportion is the sum of the percent of patients with temporary and with lasting pain relief and the second proportion is the percent of patients with no pain relief. The determination was made using eighty percent certainty to detect a true difference between the combined pain relief and no pain relief proportions at the five percent level of significance using the “Method of testing equality of two percentages.” From clinical experience, an assumption was made that sixty-six percent of the nerve block injected patients will obtain pain relief and thirty-three percent will not. Given this assumption, a minimum sample size was calculated to be forty subjects.

At step 106, sensor data of the lower back is collected from each patient. In one example, sequential recordings or scans of LASE data were collected during each of three postural tasks. The three test positions, representing increasing levels of minimal low back stress are: standing upright (Standing), standing with twenty degrees of trunk flexion (Flexion), and standing upright with arms extended forward holding a small weight of three pounds in each hand (Weighted). Three scans of the EMG electrode array can be performed at each postural position with thirty seconds rest between scans and sixty seconds rest between postural positions. The scans can be stored digitally for analysis and visual display.

The scans can be taken such that all patients in the study have a minimum of two scans per test position after screening for artifacts, high noise levels, bad scans, or faulty electrodes within the electrode array. In the described example, for every patient, each box in the six by eight grid can utilize a total of either 8 or 12 individual voltage measurements (e.g., two or three at each corner) per patient per position. The grand means of the voltage measurements are calculated using all voltage values for every box for each of the three positions. These grand means were tested for significant differences among the three patient populations. Because of multiple means comparisons, a Tukey-Kramer adjustment can be applied to insure correct ρ values for tests of significant differences between group means. This analysis can be performed, for example, using SAS (version 9.1, SAS Institute Inc, Cary, N.C.) statistical software.

Since it has been shown that the intra-examination coefficients of variation can be expected to be low, only the first scan from the series of three for each test position is used for a predictive part of the analysis. The forty-eight “boxes” with four electrode pairs in the six by eight grid can be organized into left and right side blocks based on their location in the electrode array. In accordance with an aspect of the present invention, significant differences have been determined in the LASE pattern based on whether the pain was bilateral, right sided alone or left sided alone. Combining patients from these three Permanently Improved groups with pain in different areas has been found to result in inaccurate classifications. Hence, the Permanently Improved group can be subdivided into Improved-Bilateral and Improved-Right groups based on the patient reported pain location. An Improved-Left group could also be incorporated into the classification model, but in the described example, only two patients who had back pain primarily on their left side. Accordingly, data was not available for incorporating an Improved-Left group into the model.

The goal of the analytical method was to assist the clinician by predicting, prior to intervention, whether the patient will respond positively or not to facet nerve block. It was determined that identifying patients likely to experience temporary improvement would not be of much assistance to the clinician. Thus, patients in the Temporary Improvement group can thus be assigned at the end of the prediction analysis into the Improved-Bilateral (Imp-B), Improved-Right (Imp-R), or No-Improvement (No-Imp) group.

At step 108, the subjects are classified according to data that is independent of any sensor data. The independent data can include any appropriate measurement of the patients' condition including self-reporting, other diagnostic tests, etc. In the described example, the patients can be classified into a Permanent Improvement group, a Temporary Improvement group, or a No-improvement group based on the change in their self-reported pain value after the diagnostic nerve block procedure. The patient's self reported VAS was compared with the changes in their Roland and Oswestry scores during their subsequent visits to confirm the diagnostic outcome. Also, changes in the pain location (area) on the electrode array printout were used to determine if the facet nerve block has reduced the painful area. This “boxed” CERSR data was grouped into Permanent Improvement group, Temporary Improvement group or No-Improvement group based on the patient's VAS score described above. The next step was to determine if there were differences in CERSR data between the three outcome groups to be used for prediction of the outcome of the diagnostic procedure.

At step 110, the sensor data acquired for each patient is organized to produce representative statistics of each patient category. In the described example, a database can be created for the three test positions for each of the three outcome groups. The sixty-two “box” averaged, BMI-adjusted data from six patients in each group can be used to develop the database. The remaining patient data can be used for validation analysis later. A grand mean for each of the sixty-two boxes was calculated for each position in each group. It will be appreciated that using data from just six patients per group may limit the accuracy of the database. Ideally, the database can be developed using a larger patient population to be clinically more meaningful.

In all cases it was possible to detect a significant difference in the population means between the three groups. This was due, in part to the large number of samples per mean, which resulted in estimates of the standard deviations of the population means, which were small. Repeated measurements were taken for each patient for three different positions—Standing, Flexion, and Weighted. In addition to differences in grand means between the three groups, there is also a difference in measurement variation between the three positions. Table 1 provides a summary of the pair wise comparisons of these grand means and other statistical results.

TABLE 1

| | Patient | Group | Patient | Group | P-Value of |
|---|---|---|---|---|---|
| Position | Group | Average | Group | Average | Diff. |
| Upright | Imp. | 23.0 | Not Imp. | 13.5 | <.0001 |
| | Imp. | 23.0 | Temp. Imp. | 15.5 | <.0001 |
| | Temp. Imp. | 15.5 | Not Imp. | 13.5 | .018 |
| Weighted | Imp. | 46.1 | Not Imp. | 49.1 | <.0001 |
| | Imp. | 46.1 | Temp. Imp. | 45.3 | .46 |
| | Temp. Imp. | 45.3 | Not Imp. | 49.1 | <.0001 |
| Flexion | Imp. | 31.0 | Not Imp. | 36.6 | <.0001 |
| | Imp. | 31.0 | Temp. Imp. | 35.3 | <.0001 |
| | Temp. Imp. | 35.3 | Not Imp. | 36.6 | .011 |

At step 112, the representative statistics are validated using a test set of subjects. Data associated with the test set is classified using the representative statistics representing the plurality of classes and a desired classification technique to determine the accuracy and efficiency of the classification. It will be appreciated that the test set can include subjects who were also part of the training set used to generate the representative statistics. In the described example, data from eighteen of the thirty-two patients was used to create the database and the analytical method was validated using all thirty-two patients. This method has been suggested by the SAS Institute and has been used in many preliminary studies.

In the described example, the algorithm was able to correctly predict the clinical outcome in thirty out of the thirty-two patients. It identified twenty patients as belonging to the Improved-Bilateral or Improved-Right group and twelve patients as belonging to the No-improvement group. The classification using the patient self-reported VAS scores indicated that eighteen patients belong to the Permanent Improvement group, eight to Temporary Improvement group, and six to No-improvement group. The clinical outcomes recorded in the medical charts showed that nineteen patients were considered to be Positive Facet responders (i.e., Improved group) and thirteen patients did not respond positively to the nerve block (i.e., No-improvement group). There were some discrepancies between the patients self-reported VAS obtained during the LASE procedure and those obtained in the clinical setting immediately following the nerve block—possibly due to the wearing off of the local anesthetic agent.

Overall, the exemplary classification system was able to predict the outcomes correctly for the combination of three test positions in 93.75% of the patients (compared to the clinical outcome as recorded in the medical chart). The prediction was compared with that reported in the medical chart because the current gold standard of diagnosing facet pathology is by performing fluoroscopically guided nerve blocks. It will be appreciated that valuable information can be obtained through the use of more or fewer patient positions than the three listed, but it will be appreciated that the classification can be less accurate when fewer positions are utilized. In the described example, predictions using fewer positions were considerably less accurate (e.g., 71.8% for Standing, 71.8% for Weighted and 78.1% for Flexion positions; 90.6% for combination of Standing and Flexion, 78.1% for Standing and Weighted combination and 87.5% for Flexion and Weighted combinations) than the results obtained using a combination of all three patient positions.

FIG. 5 illustrates a computer system 150 that can be employed to implement systems and methods described herein, such as based on computer executable instructions running on the computer system. The computer system 150 can be implemented on one or more general purpose networked computer systems, embedded computer systems, routers, switches, server devices, client devices, various intermediate devices/nodes and/or stand alone computer systems. Additionally, the computer system 150 can be implemented as part of the computer-aided engineering (CAE) tool running computer executable instructions to perform a method as described herein.

The computer system 150 includes a processor 152 and a system memory 154. Dual microprocessors and other multi-processor architectures can also be utilized as the processor 152. The processor 152 and system memory 154 can be coupled by any of several types of bus structures, including a memory bus or memory controller, a peripheral bus, and a local bus using any of a variety of bus architectures. The system memory 154 includes read only memory (ROM) 158 and random access memory (RAM) 160. A basic input/output system (BIOS) can reside in the ROM 158, generally containing the basic routines that help to transfer information between elements within the computer system 150, such as a reset or power-up.

The computer system 150 can include one or more types of long-term data storage 164, including a hard disk drive, a magnetic disk drive, (e.g., to read from or write to a removable disk), and an optical disk drive, (e.g., for reading a CD-ROM or DVD disk or to read from or write to other optical media). The long-term data storage can be connected to the processor 152 by a drive interface 166. The long-term storage components 164 provide nonvolatile storage of data, data structures, and computer-executable instructions for the computer system 150. A number of program modules may also be stored in one or more of the drives as well as in the RAM 160, including an operating system, one or more application programs, other program modules, and program data.

A user may enter commands and information into the computer system 150 through one or more input devices 170, such as a keyboard or a pointing device (e.g., a mouse). These and other input devices are often connected to the processor 152 through a device interface 172. For example, the input devices can be connected to the system bus 156 by one or more a parallel port, a serial port or a universal serial bus (USB). One or more output device(s) 174, such as a visual display device or printer, can also be connected to the processor 152 via the device interface 172.

The computer system 150 may operate in a networked environment using logical connections (e.g., a local area network (LAN) or wide area network (WAN) to one or more remote computers 180. The remote computer 180 may be a workstation, a computer system, a router, a peer device or other common network node, and typically includes many or all of the elements described relative to the computer system 150. The computer system 150 can communicate with the remote computers 180 via a network interface 182, such as a wired or wireless network interface card or modem. In a networked environment, application programs and program data depicted relative to the computer system 150, or portions thereof, may be stored in memory associated with the remote computers 180.

From the above description of the invention, those skilled in the art will perceive improvements, changes, and modifications. Such improvements, changes, and modifications within the skill of the art are intended to be covered by the appended claims.

Having described the invention, we claim:

1. A method for non-invasively predicting the outcome of clinical procedures comprising:
positioning a myoelectric sensor array symmetrically across a midline of a body of a patient to monitor a plurality of defined regions of the body, where each region that is not on the midline has a corresponding region on the opposite side of the midline that is approximately the same distance from the midline, such that for a given region associated a structure on a first side of the body of the patient, there is a corresponding region that is associated with a corresponding structure on a second side of the body of the patient;
receiving data representing the patient from the myoelectric sensor array;
generating a representative value for each of a plurality of defined regions within the myoelectric sensor array, where each region represents a selected portion of a area of interest; and
selecting one of a plurality of diagnosis classes for the patient according to the computed at least one value.

2. The method of claim 1, wherein positioning a myoelectric sensor array comprises positioning a strip of surface electrodes on a back of the patient.

3. The method of claim 1, wherein positioning a myoelectric sensor array comprises positioning a first strip of surface electrodes on a left arm of the patient and positioning a second strip of surface electrodes on a right arm of the patient.

4. The method of claim 1, wherein receiving data representing the patient from a myoelectric sensor array positioned on a body of the patient comprising capturing data for each of a plurality of body positions for the patient.

5. The method of claim 1, wherein each of the plurality of defined regions have an associated plurality of electrode pairs, and generating a representative value for each region comprising calculating an average of the respective potential differences of the plurality of electrode pairs.

6. The method of claim 5, wherein selecting one of a plurality of diagnosis classes for the patient comprises generating a correlation value for each of the plurality of diagnostic classes between the calculated averages for the plurality of regions and respective representative values associated with each region for the diagnostic class, wherein the representative values associated with the diagnostic class are grand mean values for each region across a training set of patients belonging to the diagnostic class.

7. The method of claim 6, wherein selecting one of a plurality of diagnosis classes for the patient further comprises:
generating a correlation value for each of the diagnosis classes for each of a plurality of patient positions to obtain a plurality of sets of correlation values, each set of correlation values representing one of the plurality of diagnostic classes;
transforming each correlation value in the set to a z parameter via a Fisher z transform to produce a set of z parameters representing each diagnosis class;
averaging across each set of z parameters to produce a composite z parameter for each diagnosis class; and
selecting the diagnosis class having the largest composite z parameter.

8. The method of claim 1, wherein positioning a myoelectric sensor array comprises positioning a set of myoelectric sensors comprising a plurality of surface electrode pairs over each of the plurality of regions.

9. A system for non-invasively predicting the outcome of a clinical procedure, comprising:
a plurality of sets of myoelectric sensors, each set of myoelectric sensors being configured to allow a plurality of surface electrode pairs associated with the set to be positioned on the skin of a patient to characterize the electrical potential of an underlying muscular structure in an associated region;
a feature extraction element that generates a representative value for each of the plurality of sets of sensors; and
a classifier that selects one of a plurality of diagnosis classes for the patient according to the computed at least one value.

10. The system of claim 9, wherein the feature extractor calculates the representative value for each region as an average of the respective potential differences of the plurality of electrode pairs.

11. The system of claim 10, wherein the feature extraction element generates a correlation value for each of the plurality of diagnostic classes between the calculated averages for the plurality of regions and respective representative values associated with each region for the diagnostic class, wherein the representative values associated with the diagnostic class are grand mean values for each region across a training set of patients belonging to the diagnostic class.

12. The system of claim 11, wherein the feature extraction element generates a correlation value for each of the diagnosis classes for each of a plurality of patient positions to obtain a plurality of sets of correlation values, with each set of correlation values representing one of the plurality of diagnostic classes, transforms each correlation value in the set to a z parameter via a Fisher z transform to produce a set of z parameters representing each diagnosis class, and averages across each set of z parameters to produce a composite z parameter for each diagnosis class, the classifier selecting the diagnosis class with the largest composite z parameter.

13. The system of claim 9, wherein each region has four associated surface electrodes, arranged as the four corners of a rectangle, and a first electrode pair of the associated plurality of electrode pairs for the region comprises an electrode positioned at a first corner and an electrode positioned at a diagonally opposing corner, a second electrode pair of the associated plurality of electrode pairs comprises an electrode positioned at a second corner and an electrode positioned at diagonally opposing corner, and a third electrode pair of the associated plurality of electrode pairs comprises the electrode positioned at the first corner and the electrode positioned at the second corner.

14. The system of claim 9, wherein plurality of sets of myoelectric sensors are configured to be distributed symmetrically around a defined midline, such that each region that is not on the defined midline has a corresponding region on the opposite side of the midline and approximately the same distance from the midline.

15. The system of claim 14, wherein the defined midline is substantially aligned with the midline of the body of the patient, such that when a given region is associated with a structure on a first side of the body of the patient, its corresponding region is associated with a structure on a second side of the body of the patient.

16. A computer readable medium comprising computer executable instructions, for non-invasively predicting outcome of facet nerve block procedures on patients experiencing lower back pain, the executable instructions comprising:
a feature extractor that receives data representing a patient from a myoelectric sensor array positioned over a lower back of the patient and generates a representative value for each of a plurality of defined regions within the myoelectric sensor array, where each region represents a selected portion of a area of interest; and
a classifier that classifies the patient into one of a permanent Improvement group, a temporary improvement group, or a no-improvement group as likely to according to the computed at least one value.

17. The computer readable medium of claim 16, wherein the feature extractor receives data representing the patient from a myoelectric sensor array positioned on a body of the patient comprising capturing data for each of a standing position, a flexion position, where the patient's trunk is bent, and a weighted position, where the patient stands upright with arms extended forward holding a weight.

18. The computer readable medium of claim 16, where the myoelectric sensor array is positioned to be symmetrical along a midline of the lower back of the patient, such that each region that is not on the midline has a corresponding region on the opposite side of the midline that is approximately the same distance from the midline, such that for a given region associated a muscular structure on a first side of the lower back of the patient, there is a corresponding region that is associated with a corresponding muscular structure on a second side of the lower back of the patient.

19. The computer readable medium of claim 16, wherein the feature extractor receives data representing a plurality of electrode pairs for each of the plurality of defined regions and generates a representative value for each region comprising calculating an average of the respective potential differences of the plurality of electrode pairs.

20. The computer readable medium of claim 19, wherein the feature extractor generates a correlation value for each of the plurality of diagnostic classes between the calculated averages for the plurality of regions and respective representative values associated with each region for the diagnostic class, wherein the representative values associated with the diagnostic class are grand mean values for each region across a training set of patients belonging to the diagnostic class.
